(19) United States
(12) Patent Application Publication
(10) Pub. No.: US 2020/0206354 A1
(43) Pub. Date: Jul. 2, 2020

(54) DRUG DELIVERY COMPOSITION

(71) Applicant: PK MED SAS, PARIS (FR)

(72) Inventors: PHILIPPE POULETTY, PARIS (FR); FRÉDÉRIQUE GUILLAMOT, GERZAT (FR)

(21) Appl. No.: 16/634,136

(22) PCT Filed: Jul. 25, 2018
(86) PCT No.: PCT/EP2018/070141

§ 371 Date: Jan. 25, 2020

(30) Foreign Application Priority Data
Jul. 25, 2017 (EP) 17305992.4

(51) Int. Cl. A61K 47/34; A61K 47/42

(52) U.S. Cl. CPC A61K 47/34; A61K 47/42

(57) ABSTRACT

The present invention relates to a new drug delivery composition comprising a polymer-drug matrix wherein both a drug and a polymer-degrading enzyme are included. The invention further relates to a process for preparing such a drug delivery composition.

### FIELD OF THE INVENTION

The present invention relates to a drug delivery composition comprising at least one drug and one polymer-degrading enzyme included, preferably embedded, in a polymer-based matrix. The present invention further relates to a process for preparing a drug delivery composition. The present invention also relates to a drug delivery device, preferably a medical device made with, or shaped from, said drug delivery composition.

### BACKGROUND OF THE INVENTION

Delivery devices or compositions for drugs are well known in the medical field. Among them, drug delivery devices have been developed that allow to release, with a more or less controlled rate, a drug in vivo. Most often, the drug is associated to a polymer, used as a vehicle for the drug. For instance, there are delivery devices composed of biodegradable polymers, wherein the drug is coated on the outer surface of the polymeric structure. Alternatively, some delivery devices are constituted of a polymeric structure, in which a drug is incorporated by use of a solvent. The use of a solvent is limited to incorporation of drug soluble in a solvent able to solubilize the polymer. For instance, drugs only soluble in water cannot be incorporated in non hydrosoluble polymers, such as the ones used for applications where specific mechanical properties are needed, such as for suture, tissue engineering, scaffold, etc. The amount of drug incorporated is also limited to solubility threshold. Moreover, small numbers of solvents are usable in the medical field. Furthermore, the process of production using solvent is low and quality critical. Indeed, process of production includes steps of drying of the solvent, and cleaning of the composition in order to ensure the total absence of any trace of solvent in the final device. Production are also generally realized in batch, each of them requiring a stringent quality control. Some other drug delivery devices are constituted of a polymeric structure comprising pores filled with a liquid permeable to the passage of the drug. However, the use of a porous polymer does not lead to a content uniformity of the drug into the polymeric structure. The use of solid drug is excluded with these devices, which further require a liquid medium or carrier for the diffusion of the drug.

It is also known how to disperse a drug into a polymer structure by way of hot melt extrusion. Hot melt extrusion allows preparing a large variety of dosage forms and formulations, such as granules, pellets, tablets, ophthalmic inserts, implants, stents or transdermal systems and shows several advantages compared to solvent-based production processes, including continuous process and the absence of use of solvent which would have to be removed up to now, using costly and time-consuming steps. However, up to now, the relationship between release rate of the drug and both quantity and nature of the polymer in the drug delivery device are not perfectly controlled. The drug delivery devices do not provide a satisfactory controllably deliver and/or a prolonged deliver period of the drug.

Accordingly, there is still a need for a drug delivery composition that can provide improved drug release thanks to a control of degradation rate of drug containing materials.

### SUMMARY OF THE INVENTION

The present invention now proposes a drug delivery composition comprising both a drug and a polymer-degrading enzyme into a polymeric structure. The polymer-degrading enzyme is able to degrade at least one polymer of the polymeric structure, leading to a more controlled degradation rate of the polymer and an improved release of the drug.

It is thereby an object of the present invention to provide a drug delivery composition, wherein said composition comprises a polymer-based matrix, at least one drug, and at least one polymer-degrading enzyme, and wherein said drug and said enzyme are included, and more preferably embedded, in said polymer-based matrix.

It is another object of the invention to provide a drug delivery composition, wherein said composition comprises a drug and a polymer-degrading enzyme included, and more preferably embedded, into a polymer-based matrix, and wherein said composition is obtainable by incorporation of said drug and said enzyme in said polymer-based matrix during heat treatment at a temperature T at which the polymer is in a partially or totally molten state.

The invention further relates to a drug delivery device made with such composition.

It is a further object of the invention to provide a process for preparing a drug delivery composition, wherein said composition comprises a polymer-based matrix, a drug, and a polymer-degrading enzyme, and wherein said process comprises incorporating said drug and said enzyme into said polymer-based matrix during heat treatment of the polymer at a temperature T at which the polymer is in a partially or totally molten state and allowing preservation of enzyme and drug activities.

The invention further relates to a drug delivery device obtainable by such process.

The invention further relates to a method of delivering a drug to a subject or organism, comprising administering to said subject or organism a drug delivery device as defined above.

The invention further relates to a method of delivering a drug to a subject or organism, comprising providing a drug, incorporating said drug with a polymer-degrading enzyme into a polymer-based matrix during heat treatment of the polymer at a temperature T at which the polymer is in a partially or totally molten state, and administering said incorporated drug to said subject or organism.

The invention also relates to a drug delivery device as defined above, for use in a method of treating a subject or organism.

The invention may be used with a large diversity of drugs and polymers and has wide applications in the medical field.

### LEGEND TO THE FIGURES

FIG. 1: PLA degradation and ibuprofen release of a drug delivery composition of the invention comprising PLA, 10% ibuprofen, and 10% of PLA-degrading enzyme, compared to PLA degradation and ibuprofen release of a control composition comprising only PLA and ibuprofen.

FIG. 2: PLA degradation and naltrexone release of a drug delivery composition of the invention comprising PLA, 8% naltrexone, and 5% of PLA-degrading enzyme, compared to PLA degradation and naltrexone release of a control composition comprising only PLA and naltrexone.

FIG. 3: PLA degradation and estradiol release of a drug delivery composition of the invention comprising PLA, 5% estradiol, and 5% of PLA-degrading enzyme, compared to PLA degradation and estradiol release of a control composition comprising only PLA and estradiol.

### DETAILED DESCRIPTION OF THE INVENTION

The present invention relates to a novel drug delivery composition comprising, or consisting essentially of a polymer-based matrix, wherein at least one enzyme able to degrade a polymer of the polymer-based matrix and at least one drug are incorporated. The drug delivery compositions of the invention show good dispersion of both the enzyme and the drug into the polymer-based matrix and allow a controlled degradation rate of at least one polymer contained in the polymer-based matrix. Depending on the use of the drug delivery composition, it is possible to adapt the polymer(s) of the polymer-based matrix, to take into account, for example, the safety of the by-products for human, and/or the quantity of drug. In addition, the adequacy between the natural degradation conditions of the polymer and the physiological properties of the target (i.e., a drug delivery device that is implanted in the body) is no more a fundamental parameter to take into account when choosing the polymer. Indeed, the enzyme favors the degradation of the polymer, even in absence of its natural degradation conditions. The enzyme also allows the use of a polymer that is not biodegradable under physiological conditions (i.e., at about 37° C. and pH 7). The invention is thus particularly adapted to polymers that are not naturally degradable or slowly degradable under physiological conditions. For instance, PLA is slowly biodegradable in human body provided that its molar mass (Mw) is low, and preferably less than 100 000 g/mol. Otherwise, its biodegradation is too slow for medical applications and its use is limited for long term applications. Furthermore, low molar mass PLA shows weak mechanical properties for medical devices that must present high resistance to pression. Up to now, it is thus not possible to use PLA for all kind of intracorporal medical devices. Thanks to the invention, PLA of high molar mass, preferably with Mw higher than 100 000 g/mol, more preferably higher than 150 000 g/mol can be used for applications that take advantages of its mechanical properties such as implants. Indeed, the incorporation of an enzyme able to degrade PLA even of high molar mass can accelerate its biodegradation. Moreover, modulation of biodegradation kinetics can be achieved thanks to variation of the amount of enzyme incorporated and/or the nature of the enzyme.

### Definitions

The present disclosure will be best understood by reference to the following definitions.

Within the context of the invention, the term “drug delivery composition” refers to any composition, in liquid, gel or solid form, comprising at least one polymer-based material, which contains at least one polymer and at least one drug to be released from the composition.

Within the context of the invention, the term “drug delivery device” refers to any item made from at least one polymer-based material, preferably in solid form, such as plastic sheet, tube, rod, profile, shape, pellet, massive block, textile, fiber, scaffold, etc., which contains at least one polymer and at least one drug to be released. More preferably, the drug delivery device is a medical device.

A “polymer” refers to a chemical compound or mixture of compounds, whose structure is constituted of multiple repeating units linked by covalent chemical bonds. Within the context of the invention, the term polymer includes natural or synthetic polymers, constituted of a single type of repeat unit (i.e., homopolymers) or of a mixture of different repeat units (i.e., heteropolymers and copolymers). Within the context of the invention, the term polymer preferably refers to thermoplastic polymer.

A “polymer-based matrix” refers to a matrix comprising, as the main ingredient, one or more polymer(s). The polymer-based matrix comprises at least 51% by weight of polymer (s), based on the total weight of the composition, preferably at least 60%, 70%, 80%, 90% or 95%. The polymer-based matrix may further comprise additional compounds, such as additives. In a particular embodiment, the polymer-based matrix comprises at least 96%, 97%, 98% or 99% by weight of polymer, based on the total weight of the composition.

A “drug” refers to any substance that is biologically active, i.e., that may have an impact on a living organism, including mammal, avian, virus, fungi and microorganisms. Notably, the term drug encompasses active substances, mineral or organic, that may have a prophylactic or therapeutic activity on a mammal, substances with antifungal and/or antimicrobial activity, etc. For instance, the drug is an active agent, such as pharmaceutical agent, Traditional Chinese Medicine, antibiotic, anti-cancer agent, anti-viral agent, anti-inflammatory agent, hormone, growth factor, etc., an antigen, a vaccine, an adjuvant, etc. The drug may also consist on a cosmetic agent.

As used herein, the term “by weight” refers to the ratio based on the total weight of the considered composition or product.

In the context of the invention, the term “about” refers to a margin of +/−5%, preferably of +/−1%, or within the tolerance of a suitable measuring device or instrument.

### Polymer-Based Matrix

The present invention relates to a drug delivery composition made of a polymeric material. More particularly, the polymeric material is constituted of a polymer-based matrix that is shaped into the desired form, depending on the destination of the composition (e.g., the nature of the medical device). For instance, the device obtained from such composition is shaped as suture fibers, stent, prosthesis, patch, screw or bone plate, intra-uterine device, scaffold, implant, pump, etc.

Advantageously, both a drug to be released and a polymer-degrading enzyme that is able to degrade at least one polymer of the polymer-based matrix are added to said polymer-based matrix, so that they are included, and preferably embedded into the matrix. Advantageously, both the drug and enzyme are homogeneously embedded in the polymer-based matrix. In the context of the invention “homogeneously embedded” means that the drug and enzyme are uniformly distributed in the polymer-based matrix. Such homogeneity of the distribution in the polymer-based matrix leads to a final drug-delivery composition that presents a homogenous reparation of drug and enzyme, allowing thereby a controlled release of the drug. Such homogeneous distribution may be obtained e.g., by heating the polymer-based matrix until it is at least partially molten to allow incorporation into the molten composition of the drug and enzyme. The final drug delivery composition is advantageously in a solid state. However, it is possible to provide a drug-delivery composition that is in a molten or even liquid state.

The polymer-based matrix may be prepared from various polymers. Preferably, the polymer-based matrix comprises at least one polymer chosen among polyesters, polyethers or ester-ether copolymers. The polyester may be selected e.g., from polylactic acid (PLA), poly(L-lactic acid) (PLLA), poly(D-lactic acid) (PDLA), poly(D,L-lactic acid) (PDLLA), stereocomplex PLA (scPLA), polyhydroxy alkanoate (PHA), Poly(3-hydroxybutyrate) (P(3HB)/PHB), Poly(3-hydroxyvalérate) (P(3HV)/PHV), Poly(3-hydroxyhexanoate) (P(3HHx)), Poly(3-hydroxyoctanoate) (P(3HO)), Poly(3-hydroxydécanoate) (P(3HD)), Poly(3-hydroxybutyrate-co-3-hydroxyvalérate) (P(3HB-co-3HV)/PHB V), Poly(3-hydroxybutyrate-co-3-hydroxyhexanoate) (P(3HB-co-3HHx)/(PHBHHx)), Poly(3-hydroxybutyrate-co-5-hydroxyvalerate) (PHB5HV), Poly(3-hydroxybutyrate-co-3-hydroxypropionate) (PHB3HP), Polyhydroxybutyrate-co-hydroxyoctonoate (PHBO), polyhydroxybutyrate-co-hydroxyoctadecanoate (PHBOd), Poly(3-hydroxybutyrate-co-3-hydroxyvalerate-co-4-hydroxybutyrate) (P(3HB-co-3HV-co-4HB)), polyglycolic acid (PGA), polybutylene succinate (PBS), polybutylen succinate adipate (PBSA), polybutylen adipate terephthalate (PBAT), polycaprolactone (PCL), poly(ethylene adipate) (PEA) or copolymers thereof such as poly(lactic-co-glycolic acid) copolymers (PLGA) and blends/mixtures of these materials. The polyethers may be selected e.g., from polyethylene glycol (PEG), preferably PEG with molecular mass above 600 g/mol, polyethylene oxide (PEO), or copolymers and blends/mixtures thereof. The ester-ether copolymers may be selected e.g., from polydioxanone (PDS).

In particular, the polymer-based matrix comprises at least one polymer selected from polymers that are not naturally degradable under physiological conditions, i.e. that do not lead to any degradation into monomers and/or oligomers under physiological conditions in less than 10 years. The use of the enzyme in the drug delivery composition enable to initiate the degradation of such polymer in less than 10 years. In another particular embodiment, the polymer-based matrix comprises at least one polymer selected from polymers that are partially degradable under physiological conditions, i.e. that do not lead to a complete degradation into monomers and/or oligomers under physiological conditions in less than 10 years, preferably less than 5 years, more preferably less than 2 years. In such case, the use of the enzyme in the drug delivery composition enable to accelerate the degradation process of the polymer.

In a particular embodiment, the polymer-based matrix comprises at least one polymer selected from polylactic acid (PLA), polybutylene adipate terephthalate (PBAT), polyhydroxyalkanoate (PHA), polyglycolic acid (PGA), polybutylene succinate (PBS), polycaprolactone (PCL), poly(ethylene adipate) (PEA), dextrane, gelatin, starch, cellulose and its derivatives, poly butylene succinate adipate (PBSA), polydioxanone (PDS), polyethylene glycol (PEG), preferably PEG with molecular mass above 600 g/mol, polyethylene oxide (PEO) or copolymers, and blends/mixtures thereof.

In another particular embodiment, the polymer-based matrix comprises at least one polymer with a molecular mass in weight (Mw) greater than 100 000 g/mol.

In a further particular embodiment, the polymer-based matrix comprises PLA. Particularly, such PLA has a Mw greater than 100 000 g/mol, preferably greater than 150 000 g/mol. In a particular embodiment, the polymer-based matrix comprises PLA with Mw of 180 000 g/mol. Such polymer-based matrix may further comprise at least one additional polymer, preferably selected from, polybutylene adipate terephthalate (PBAT), polyhydroxyalkanoate (PHA), polyglycolic acid (PGA), polybutylene succinate (PBS), polycaprolactone (PCL), poly(ethylene adipate) (PEA), dextrane, gelatin, starch, cellulose and its derivatives, and blends/mixtures thereof, more preferably from PBAT or PCL. Alternatively, the polymer-based matrix contains PLA as the only polymer, preferably PLLA and/or PDLA.

In an embodiment, the polymer-based matrix comprises lactic acid copolymers, preferably selected from PLA-based heteropolymers, more preferably selected from poly(lactic-co-glycolic acid) copolymers (PLA-co-PGA or PLGA), poly(lactic-co-caprolactone) copolymers (PLA-co-PCL), poly(lactic-co-ethyleneglycol) copolymers (PLA-co-PEG), poly(lactic-co-ethylene oxide) copolymers (PLA-co-PEO) or grafted PLA (PLA-g-gelatine).

In another particular embodiment, the polymer-based matrix contains PCL. Such polymer-based matrix may further comprise at least one additional polymer, preferably selected from polybutylene adipate terephthalate (PBAT), polyhydroxyalkanoate (PHA), polyglycolic acid (PGA), polybutylene succinate (PBS), polylactic acid (PLA), poly(ethylene adipate) (PEA), dextrane, gelatin, starch, cellulose and its derivatives, and blends/mixtures of these polyesters or copolymers. Alternatively, the polymer-based matrix contains PCL as the only polymer.

In another particular embodiment, the polymer-based matrix contains PGA. Such polymer-based matrix may further comprise at least one additional polymer, preferably selected from polybutylene adipate terephthalate (PBAT), polyhydroxyalkanoate (PHA), polycaprolactone (PCL), polybutylene succinate (PBS), polylactic acid (PLA), poly(ethylene adipate) (PEA), dextrane, gelatin, starch, cellulose and its derivatives, and blends/mixtures of these polyesters or copolymers. Alternatively, the polymer-based matrix contains PGA as the only polymer.

The choice of the polymer(s) may be adjusted by the skilled artisan, depending on the destination and use of the drug delivery composition. For example, for a medical device made with said composition and that dedicated to be implanted into a mammal body, the polymers should preferably disintegrate innocuously or break down as safe unit structures. Indeed, in case of medical devices that must be implanted into a body, it may be interesting to take into account the molar mass of the monomers (produced from polymer disintegration) to be sure that they can be biologically eliminated (e.g., renal elimination, hepatic elimination, etc.).

According to the invention, the polymer-based matrix may further contain additives such as acid neutralizing agents, preferably selected from carbonate salts, calcium phosphate, hydrotalcite, talc, mica, and clay.

### Polymer-Degrading Enzyme

According to the invention, the drug delivery composition contains at least one polymer-degrading enzyme that is able to degrade at least one polymer of the polymer-based matrix. The incorporation of a polymer-degrading enzyme allows to increase the degradability of the polymer-based matrix and thus provides improved release of the drug.

In a particular embodiment, the drug delivery composition comprises one or more enzymes that can degrade all polymers contained in the polymer-based matrix.

For instance, in a particular embodiment, the polymer-based matrix is composed of a single polymer and the drug delivery composition contains one or more enzymes that degrade said polymer.

In another particular embodiment, the polymer-based matrix comprises two different polymers and the drug delivery composition contains one or more enzymes that degrade both polymers.

In another particular embodiment, the polymer-based matrix comprises two different polymers and the drug delivery composition contains one or more enzymes that degrade only one of said polymers.

In the context of the invention, a “polymer-degrading enzyme” refers to an enzyme suitable for hydrolyzing chemical bonds between monomers of at least one polymer. Preferably, the polymer-degrading enzyme is suitable for depolymerizing at least one polymer of the drug delivery device up to oligomers and/or monomers. Advantageously, the oligomers and/or monomers are innocuous for the human body. In a particular embodiment, the degrading enzyme is able to depolymerize the polymer of the drug delivery composition up to monomers. Such embodiment may be of particular interest for medical devices that are implanted into a body, in order to favor the biological elimination of the by-products of the medical device.

The polymer-degrading enzyme may be selected depending on the nature of the polymer(s). Preferably, the polymer-degrading enzyme is suitable for depolymerizing at least one polyester of the drug delivery device up to oligomers and/or monomers.

In a particular embodiment, the degrading enzyme is suitable for depolymerizing at least one polymer of the drug delivery device up to oligomers and/or monomers under physiological conditions. Preferably, the degrading enzyme is active at 37° C. and/or at pH between 7 and 7.5. In another particular embodiment, the degrading enzyme is selected from an enzyme having an optimum pH, close to physiological pH, i.e. a pH between 6 and 8.

The degrading enzyme is preferably selected from cutinases (EC 3.1.1.74), lipases (EC 3.1.1.3), esterases, carboxylesterases (EC 3.1.1.1), serine proteases (EC 3.4.21.64), proteases, and oligomer hydrolases.

Serine proteases (such as Proteinase K from Tritirachium album or PLA depolymerase from Amycolatopsis sp., Actinomadura keratinilytica, Laceyella sacchari LP175, Thermus sp., or Bacillus licheniformis or any reformulated commercial enzymes known for degrading PLA such as Savinase®, Esperase®, Everlase® or any enzymes from the family of the subtilisin CAS 9014-01-1 or any functional variant thereof), lipases (such as the one from Candida antarctica or Cryptococcus sp or Aspergillus niger), and/or esterases (such as the one from Thermobifida halotolerans) or variants thereof may be used for depolymerizing a drug delivery composition containing polylactic acid (PLA).

Cutinases (such as the one from Thermobifida fusca or Thermobifida alba or Fusarium solani pisi) and lipases (such as lipase PS from Burkholderia cepacia) or variants thereof may be used for depolymerizing a drug delivery composition containing PCL.

Proteases (such as carboxypeptidase, clostridiopeptidase, alpha-chymotrypsin, trypsin or ficin) or esterases or variants thereof may be used for depolymerizing a drug delivery device containing PGA.

In a preferred embodiment, the invention thus relates to a drug delivery composition, such as a drug delivery device comprising a PLA-based matrix, a drug, and a PLA-degrading enzyme preferably selected from a serine-protease, a lipase, or an esterase.

In another preferred embodiment, the invention relates to a drug delivery composition, such as a drug delivery device comprising a PCL-based matrix, a drug, and a PCL-degrading enzyme preferably selected from a cutinase or a lipase.

In another preferred embodiment, the invention relates to a drug delivery composition, such as a drug delivery device comprising a PGA-based matrix, a drug, and a PGA-degrading enzyme preferably selected from a protease or an esterase.

### Drug

According to the invention, the drug is chosen to act on a biological target. In the context of the invention, a “biological target” refers to any biological entity that may be directly or indirectly impacted by the drug. The biological target may be a whole body, an organ, a tissue, specific cells, etc., of an animal, such as a mammal or an avian, a microorganism, a virus, etc.

Preferably, the drug is selected from chemicals, pharmaceutical compound, nutraceutical compound, amino acids, peptides, proteins, polysaccharides, lipid derivatives, antibiotics, analgesics, vaccines, vaccine adjuvants, anti-inflammatory agents, anti-tumor agents, hormones, cytokines, anti-fungal agents, anti-viral agents, anti-bacterial agents, anti-diabetics, steroids, vitamins, pro-vitamins, antioxidants, mineral salts, trace elements, specific enzyme inhibitor, growth stimulating agent, immunosuppressors, immuno-modulators, anti-hypertensive drugs, anti-arythmic drugs, inotropic drugs, addiction therapy drugs, anti-epileptic drugs, anti-aging drugs, drugs to treat neuropathies or pain, hypolipemic drugs, anti-coagulants, antibodies or antibody fragments, antigens, anti-depressant or psychotropic agents, neuro-modulators, drugs for treating a disease selected from brain disease, liver disease, pulmonary disease, cardiac disease, gastric disease, intestine disease, ovary disease, testis disease, urological disease, genital disease, bone disease, muscle disease, endometrial disease, pancreatic disease and/or renal disease, ophthalmic drugs, anti-allergic agents, contraceptive or luteinizing agents, enzymes, Traditional Chinese Medicines, nutrients, cosmetics and mixtures of at least two of these drugs.

In a particular embodiment, the drug is selected from chemicals, pharmaceutical compound, amino acids, peptides, proteins, antibiotics, analgesics, vaccines, vaccine adjuvants, anti-inflammatory agents, anti-tumor agents, hormones, cytokines, anti-fungal agents, anti-viral agents, anti-bacterial agents, anti-diabetics, steroids, specific enzyme inhibitor, growth stimulating agent, immunosuppressors, immuno-modulators, anti-hypertensive drugs, anti-arythmic drugs, inotropic drugs, addiction therapy drugs, anti-epileptic drugs, anti-aging drugs, drugs to treat neuropathies or pain, hypolipemic drugs, anti-coagulants, antibodies or antibody fragments, antigens, anti-depressant or psychotropic agents, neuro-modulators, drugs for treating a disease selected from brain disease, liver disease, pulmonary disease, cardiac disease, gastric disease, intestine disease, ovary disease, testis disease, urological disease, genital disease, bone disease, muscle disease, endometrial disease, pancreatic disease and/or renal disease, ophthalmic drugs, anti-allergic agents, contraceptive or luteinizing agents, enzymes and mixtures of at least two of these drugs.

In a particular embodiment, the drug is chosen among compounds having therapeutic or prophylactic purposes in a mammal, and more particularly in a human.

In a particular embodiment, the drug is chosen among compounds having a denaturation temperature below 120° C., preferably below 100° C. In the context of the invention, the denaturation temperature corresponds to the temperature at which half of the drug loses its activity. Generally, the denaturation temperature is preferably above 50° C.

In another particular embodiment, the drug has a molecular mass above 10 kDa, preferably above 14 kDa. In another embodiment, the drug has a molecular mass above 15 kDa.

In a particular embodiment, the drug is chosen from a protein having a molecular mass above 10 kDa such as lysozyme. In another particular embodiment, the drug is chosen from a protein having a molecular mass above 50 kDa, preferably above 100 kDa such as antibodies. In another particular embodiment, the drug is chosen from enzyme having a molecular mass above 30 kDa, preferably above 50 kDa such as lipase. In another particular embodiment, the drug is chosen from a hormone having a molecular mass above 9 kDa such as insulin or parathyroid hormone. In another particular embodiment, the drug is a growth hormone having a molecular mass above 20 kDa. In another particular embodiment, the drug is a hormone having a molecular mass above 30 kDa such as erythropoietin.

### Drug Delivery Composition

It is the purpose of the invention to provide new drug delivery compositions allowing release, preferably in a controlled rate, of a drug that is incorporated into said delivery composition.

In a particular embodiment, the drug delivery composition is a pharmaceutical composition. Such pharmaceutical composition may be in the form of a tablet, gel, coating, particles, or microbeads.

It is also a purpose of the invention to provide a new drug delivery device allowing to release, preferably in a controlled rate, a drug that is included into said delivery device. Accordingly, the composition of the invention may advantageously be used to shape a drug delivery device, more particularly a medical device.

Such medical device may be in the form of an implant, film, stent, leaflet, valve, coil, scaffold, dressing, rod, patch, fibers, suture fibers, screw, bone plate or implant, bone cement and prostheses.

In a particular embodiment, the drug delivery composition comprises
from 51 to 99.98% by weight of polymer-based matrix,
from 0.01 to 49% by weight of a drug, and
from 0.01 to 30% by weight of the polymer-degrading enzyme.

In a particular embodiment, the drug delivery composition comprises
from 50 to 99.98% by weight of polymer-based matrix,
from 0.01 to 49.99% by weight of a drug, and
from 0.01 to 30% by weight of the polymer-degrading enzyme.

In a preferred embodiment, the drug delivery composition comprises
from 60 to 99.98% by weight of polymer-based matrix,
from 0.01 to 39.99% by weight of a drug, and
from 0.01 to 20% by weight of the polymer-degrading enzyme.

In a preferred embodiment, the drug delivery composition comprises
from 60 to 99.98% by weight of polymer-based matrix,
from 0.01 to 39% by weight of a drug, and
from 0.01 to 20% by weight of the polymer-degrading enzyme.

For instance, the drug delivery composition comprises 90% by weight of polymer-based matrix, 5% by weight of a drug, and 5% by weight of the polymer-degrading enzyme.

Alternatively, the drug delivery composition comprises 85% by weight of polymer-based matrix, 10% by weight of a drug, and 5% by weight of the polymer-degrading enzyme.

Alternatively, the drug delivery composition comprises 80% by weight of polymer-based matrix, 5% by weight of a drug, and 15% by weight of the polymer-degrading enzyme.

Alternatively, the drug delivery composition comprises 80% by weight of polymer-based matrix, 10% by weight of a drug, and 10% by weight of the polymer-degrading enzyme.

Alternatively, the drug delivery composition comprises 70% by weight of polymer-based matrix, 20% by weight of a drug, and 10% by weight of the polymer-degrading enzyme.

Alternatively, the drug delivery composition comprises 60% by weight of polymer-based matrix, 30% by weight of a drug, and 10% by weight of the polymer-degrading enzyme.

In a particular embodiment, the polymer-based matrix consists on PLA, the polymer-degrading enzyme is a PLA depolymerase, such as proteinase K or a serine protease, and the drug is selected from bone regenerative enzymes, anti-inflammatory agents (e.g., ibuprofene), analgesic (e.g., paracetamol, morphine), anti-diabetics (e.g., insulin), hormone (e.g., progesterone), cytokine, monoclonal antibody, antigen, contraceptive agent, anti-tumor agent, and anti-infectious agent.

In a particular embodiment, the invention relates to a drug delivery composition, such as a drug delivery device comprising a PLA-based matrix, a drug selected from a pharmaceutical compound useful to manage alcohol or opioid dependence, preferably naltrexone, and a PLA-degrading enzyme, preferably a serine-protease. In a particular embodiment, the drug delivery composition comprises from 74.99 to 99.98% by weight of PLA-based matrix, from 0.01 to 15% by weight of naltrexone, and from 0.01 to 15% by weight of the PLA-degrading enzyme (e.g., serine protease). In another particular embodiment, the drug delivery composition comprises from 51 to 80% by weight of PLA-based matrix, from 19.99 to 48.99% by weight of naltrexone, and from 0.01 to 20% by weight of the PLA-degrading enzyme (e.g., serine protease). In a particular embodiment, the drug delivery composition comprises 87%, +/−10%, by weight of PLA with molecular weight (Mw) 180 000 g/mol, 8%, +/−10%, by weight of naltrexone hydrochloride and 5%, +/−10%, by weight of a formulation of Savinase®, based on the total weight of the drug delivery composition.

In a particular embodiment, the invention thus relates to a drug delivery composition, such as a drug delivery device comprising a PLA-based matrix, a nonsteroidal anti-inflammatory drug, preferably ibuprofen, and a PLA-degrading enzyme, preferably a serine-protease. In a particular embodiment, the drug delivery composition comprises from 70 to 99.98% by weight of PLA-based matrix, from 0.01 to 20% by weight of ibuprofen, and from 0.01 to 10% by weight of the PLA-degrading enzyme (e.g., serine protease). In another particular embodiment, the drug delivery composition comprises from 51 to 90% by weight of PLA-based matrix, from 9.99 to 48.99% by weight of ibuprofen, and from 0.01 to 20% by weight of the PLA-degrading enzyme (e.g., serine protease). In a particular embodiment, the drug delivery composition comprises 80%, +/−10%, by weight of PLA with molecular weight (Mw) 180 000 g/mol, 10%, +/−10%, by weight of S-Ibuprofen and 10%, +/−10%, by weight of a formulation of Savinase®.

In a particular embodiment, the invention thus relates to a drug delivery composition, such as a drug delivery device comprising a PLA-based matrix, a hormone, preferably estradiol, and a PLA-degrading enzyme, preferably a serine-protease. In a particular embodiment, the drug delivery composition comprises from 85 to 99.98% by weight of PLA-based matrix, from 0.01 to 10% by weight of estradiol, and from 0.01 to 10% by weight of the PLA-degrading enzyme (e.g., serine protease). In another particular embodiment, the drug delivery composition comprises from 51 to 90% by weight of PLA-based matrix, from 9.99 to 48.99% by weight of estradiol, and from 0.01 to 20% by weight of the PLA-degrading enzyme (e.g., serine protease). In a particular embodiment, the drug delivery composition comprises 90%, +/−10%, by weight of PLA with Mw 180 000 g/mol), 5%, +/−5%, by weight of estradiol and 5%, +/−5%, by weight of a formulation of Savinase®, based on the total weight of the drug delivery composition.

In a particular embodiment, the invention thus relates to a drug delivery composition, such as a drug delivery device comprising a PLA-based matrix, a drug selected from a protein preferably lysozyme, and a PLA-degrading enzyme, preferably a serine-protease. In a particular embodiment, the drug delivery composition comprises from 70 to 99.98% by weight of PLA-based matrix, from 0.01 to 20% by weight of lysozyme, and from 0.01 to 10% by weight of the PLA-degrading enzyme (e.g., serine protease). In another particular embodiment, the drug delivery composition comprises from 50 to 99.98% by weight of PLA-based matrix, from 0.01 to 49.99% by weight of lysozyme, and from 0.01 to 10% by weight of the PLA-degrading enzyme (e.g., serine protease).

In a particular embodiment, the drug is formulated in a polymer carrier, preferably PCL and is introduced in a form of a masterbatch. The invention thus relates to a drug delivery composition, such as a drug delivery device comprising a PLA-based matrix, a drug selected from a protein preferably lysozyme and formulated in PCL, and a PLA-degrading enzyme, preferably a serine-protease. In a particular embodiment, the drug delivery composition comprises from 50 to 99.97% by weight of PLA-based matrix, from 0.01 to 20% by weight of lysozyme, from 0.01 to 20% by weight of PCL, and from 0.01 to 10% by weight of the PLA-degrading enzyme (e.g., serine protease). In a particular embodiment, the drug delivery composition comprises 70%, +/−10%, by weight of PLA with Mw 180 000 g/mol), 10%+/−10% by weight of PCL, 10%, +/−10%, by weight of lysozyme and 10%, +/−10%, by weight of a formulation of Savinase®, based on the total weight of the drug delivery composition. In a particular embodiment, the invention thus relates to a drug delivery composition, such as a drug delivery device comprising a PLGA-based matrix, or PLA/PGA based matrix, a drug, and a PGLA-degrading enzyme or PLA-degrading enzyme, or PGA-degrading enzyme or mix thereof. In a particular embodiment, the drug delivery composition comprises from 70 to 99.98% by weight of PLGA-based matrix, or PLA/PGA based matrix, from 0.01 to 20% by weight of drug, and from 0.01 to 10% by weight of the PGLA-degrading enzyme or PLA-degrading enzyme, or PGA-degrading enzyme or mix thereof. In another particular embodiment, the drug delivery composition comprises from 50 to 99.98% by weight of PLGA-based matrix, or PLA/PGA based matrix, from 0.01 to 49.99% by weight of drug, and from 0.01 to 10% by weight of PGLA-degrading enzyme or PLA-degrading enzyme, or PGA-degrading enzyme or mix thereof.

In another particular embodiment, the polymer-based matrix consists on PCL, the polymer-degrading enzyme is a lipase PS, and the drug is selected from bone regenerative enzymes, anti-inflammatory agents (e.g., ibuprofene), analgesic (e.g., paracetamol, morphine), anti-diabetics (e.g., insulin), hormone (e.g., progesterone), cytokine, monoclonal antibody, antigen, contraceptive agent, anti-tumor agent, and anti-infectious agent. The present invention thus relates to a drug delivery composition comprising comprises from 70 to 99.98% by weight of PCL-based matrix, from 0.01 to 20% by weight of an enzyme like lysozyme, and from 0.01 to 10% by weight of the PCL-degrading enzyme. In another particular embodiment, the drug delivery composition comprises from 50 to 99.98% by weight of PCL-based matrix, from 0.01 to 49.99% by weight of lysozyme, and from 0.01 to 10% by weight of the PCL-degrading enzyme.

In another particular embodiment, the polymer-based matrix consists on PGA, the polymer-degrading enzyme is an esterase, and the drug is selected from bone regenerative enzymes, anti-inflammatory agents (e.g., ibuprofene), analgesic (e.g., paracetamol, morphine), anti-diabetics (e.g., insulin), hormone (e.g., progesterone), cytokine, monoclonal antibody, antigen, contraceptive agent, anti-tumor agent, and anti-infectious agent.

The present invention interestingly allows to incorporate a drug within a polymer-based matrix at a high concentration and particularly above its solubility threshold in classical solvents used for drug incorporation, such as chloroform or dichloromethane. Solubility threshold is the maximum concentration for a drug to be soluble in a solvent at ambient temperature. Indeed, up to now, a drug is introduced in a polymer-based matrix by use of a solvent, which impacts the final concentration of the drug within the polymer-based matrix. According to the invention, it is now possible to provide drug delivery composition, wherein the concentration of the drug is greater than the concentration obtainable with a solvent-based process. For instance, the ratio drug/polymer-based matrix may be between 0.5 and 2.3, and notably 1. Alternatively, the ratio drug/polymer-based matrix may be between 0.05 and 0.7.

The drug may be introduced in the polymer-based matrix under solid form (such as powder) or liquid form, when said polymer-based matrix is in partially or totally molten state. Furthermore, according to the invention, it is possible to incorporate an aqueous composition comprising water and a water-soluble drug. This is particularly adapted for producing a drug delivery composition comprising a drug insoluble in classical solvents but soluble in water. According to the invention, the aqueous composition may be incorporated in the polymer-based matrix in totally or partially molten state, for instance during an extrusion process.

### Drug Delivery Composition Preparation Process

The present invention also relates to a process for preparing a drug delivery composition, wherein said composition comprises a polymer-based matrix, a drug, and a polymer-degrading enzyme, and wherein said process comprises incorporating said drug and said enzyme into said polymer-based matrix during heat treatment of the polymer at a temperature T at which the polymer is in a partially or totally molten state. Preferably, the drug and enzyme are incorporated at a temperature T between 50° C. and 200° C., preferably between 60° C. and 180° C., more preferably between 70° C. and 160° C. The temperature T can be adapted by a person skilled in the art depending on the polymer and/or drug and/or enzyme of the drug delivery composition.

In a particular embodiment, the drug and enzyme are incorporated simultaneously, preferably at a temperature T which is above the glass transition temperature (Tg) of the polymer, preferably at or above the melting temperature of the polymer.

In another embodiment, the drug and the enzyme are incorporated sequentially.

For instance, the enzyme is incorporated first, preferably at a temperature T which is above the glass transition temperature (Tg) of the polymer, preferably at or above the melting temperature of the polymer, and the drug is subsequently incorporated, preferably at a temperature T between the glass transition temperature (Tg) and the melting temperature of said polymer.

Alternatively, the drug is incorporated first, preferably at a temperature T which is above the glass transition temperature (Tg) of the polymer, preferably at or above the melting temperature of the polymer, and the enzyme is subsequently incorporated, preferably at a temperature T between the glass transition temperature (Tg) and the melting temperature of said polymer.

Advantageously, the heat treatment is selected from extrusion, internal mixing, co-kneading, injection-molding, thermoforming, rotary molding, compression, calendering, ironing, coating, stratification, expansion, pultrusion, extrusion blow-molding, extrusion-swelling, compression-granulation and 3D printing such as fused deposition modelling, selective laser sintering or binder jetting, preferably an extrusion and 3D printing. Depending on the chosen heat treatment, the polymer-based matrix may be both melted with enzyme and drug and shaped into the desired form.

In a preferred embodiment the heat treatment is an extrusion, advantageously performed in an extruder. For instance, the extruder may be a multi-screw extruder, preferably a twin-screw extruder, more preferably a co-rotative twin-screw extruder.

In a preferred embodiment, the residence time of the enzyme and/or drug in the extruder is comprised between 5 seconds and 3 minutes, preferably is less than 2 minutes, more preferably less than 1 minute. When the polymer-based matrix comprises a polymer with a melting temperature below 180° C., the residence time of the mixture in the extruder is preferably less than 2 minutes. Residence time depends on the process and the polymer-based matrix and may be easily adjusted by the person skilled in the art.

Both the enzyme and the drug may be introduced in the extruder in a solid form, such as a powder, or liquid form, such as a liquid formulation. Advantageously, the enzyme and/or the drug are introduced at a late stage of the heat treatment, and more particularly once the polymer-based matrix is in a partially or totally molten state. Thus, the exposure to elevated temperature is reduced. Preferably, the residence time of both the enzyme and the drug in the extruder is half as long as the residence time of the polymer-based matrix, or less.

Enzyme and drug can be formulated in any support known by the person skilled in the art. A single formulation containing both enzyme and drug can be used.

In a particular embodiment, enzyme and/or drug are formulated in a polymer carrier, preferably in a polymer with a melting temperature below 140° C. Preferably, the enzyme and/or drug are introduced in a form of a masterbatch. According to a particular embodiment, said masterbatch is prepared by (i) extruding a carrier polymer and (ii) introducing the drug and/or the enzyme during extrusion of the carrier polymer. The masterbatch can thus be introduced within a polymer-based matrix to obtain the drug delivery composition according to the invention. This embodiment of the invention is of particular interest to control with more accuracy the final dosage and homogeneity of the drug into the drug-delivery composition/device.

Preferably, said carrier polymer has a melting temperature below 140° C. and is preferably selected from polycaprolactone (PCL), poly butylene succinate adipate (PBSA), polybutylene adipate terephthalate (PBAT), polydioxanone (PDS), polyhdroxyalkanoate (PHA), polylactic acid (PLA), polyglycolic acid (PGA), polyethylene glycol (PEG), preferably PEG with molecular mass above 600 g/mol, polyethylene oxide (PEO) or copolymers. In a particular embodiment, the enzyme and/or drug are formulated in a polymer carrier selected from PCL and is introduced in a form of a masterbatch.

In another particular embodiment, the drug and/or the enzyme are formulated within an aqueous solvent, preferably water, before to be introduced in the polymer-based matrix.

It is another object of the invention to provide a medical drug device obtained from a process comprising a step of incorporating a drug and an enzyme having a polymer-degrading activity into a polymer-based matrix, and wherein such step is performed by a heat treatment of the polymer at a temperature T at which the polymer is in a partially or totally molten state.

### EXAMPLES

### Example 1—Drug Delivery Composition of the Invention Comprising Ibuprofen, PLA and PLA-Degrading Enzyme

A drug delivery composition of the invention was prepared by mixing 80% by weight of micronized polymer of polylactic acid (Ingeo™ Biopolymer 4043D from NatureWorks, molecular weight (Mw) 180 000 g/mol), 10% by weight of 5-Ibuprofen powder (from Sigma-Adrich reference 375160) and 10% by weight of a formulation of Savinase® under a powder form, based on the total weight of the drug delivery composition. Savinase® is an enzyme from Novozymes, that is known to have the ability to degrade PLA (Degradation of Polylactide by commercial proteases; Y. Oda, A. Yonetsu, T. Urakami and K. Tonomura; 2000).

The formulation of Savinase® under a powder form was obtained as follow: a liquid formulation was obtained by ultrafiltation and diafiltration of the commercial Savinase® 16 L (diafiltration factor about 100) on 3.5 Kd membrane to obtain a concentrated liquid composition and to remove some polyols present in the commercial solution. Arabic gum (INSTANT GUM AA—NEXIRA) was added and the composition obtained was then dried by freeze drying in order to obtain a solid composition comprising about 33% by weight of enzyme, 15.7% by weight of arabic gum, 0.5% by weight of water and 50.8% by weight of polyols (glycerol, propylene glycol) and other additives, based on the total weight of the solid composition.

The mix was then extruded using a twin-screw extruder (Thermo Scientific HAAKE Minilab II) to incorporate ibuprofen and Savinase® into PLA. A control composition without Savinase® was also prepared. The twin screw extruder was used at 80 rpm with a manual loading of the composition.

A mix composed of 4.0 g of PLA, 0.5 g of S-Ibuprofen and 0.5 g of solid composition comprising Savinase® has been extruded at 155° C. to produce a drug delivery composition of the invention. The control composition without Savinase® comprises 4.5 g of PLA and 0.5 g of 5-Ibuprofen.

The degradation of PLA and the release of ibuprofen were analyzed by UHPLC for titration of lactic acid and ibuprofen, using methods described below. Compositions were cut in small fragments with a cutting pliers. About 100 mg of these compositions were introduced in a dialysis tubing cellulose membrane (cut off 14 000 Da—From Sigma-Aldrich) with 3 mL of Tris-HCl buffer 0.1 M pH 8. The dialysis tubings were then introduced in 50 mL of Tris-HCl buffer 0.1 M pH 8 and incubated at 37° C. during several days. Samples were taken off at different times during the degradation of the compositions.

### UHPLC Method Used for Lactic Acid Titration:

An Ultimate 3000 HPLC system (Thermofisher Scientific) equipped with a Refractive Index Detector Shodex RI-101 Analytical and a Phenomenex RFQ-Fast Acid H+(8%), 7.8×100 mm, 8 μm column were used. The column was controlled to a temperature of 60° C. The mobile phase was H₂SO₄ 5 mm with of flow rate of 0.75 mL/min. Lactic acid (LA) powder was accurately weighed and dissolved in water to give 10 g/L solution. Subsequent dilutions were made with water to get concentrations of 0.5-5 g/L of LA. The standard solutions prepared as above were injected (20 μL) in the same conditions of samples. The peak areas of the lactic acid concentration were calculated. The regression of the LA concentration over the peak areas was obtained and used to estimate the amount of LA released from the polymer.

### UHPLC Method Used for Ibuprofen Titration:

An Ultimate 3000 HPLC system (Thermofisher Scientific) equipped with Diode Array Detector (DAD-3000(RS)) and a Phenomenex Kinetex EVO C18, LC Column 100×2.1 mm, 2.6 μm with a pore size of 100 Å. The column was controlled to a temperature of 50° C. The mobile phase was a mix with 38% acetonitrile and 62% of 20 mM K2HPO4 buffer pH3 with phosphoric acid with flow rate of 0.75 mL/min. 5-Ibuprofen powder was accurately weighed and dissolved in mobile phase to give 400 μg/mL solution. Subsequent dilutions were made with mobile phase to get concentrations of 23-400 μg/mL. The standard solutions prepared as above were injected (20 μL) in the same conditions of samples. The peak areas of the ibuprofen concentration were calculated. The regression of the ibuprofen concentration over the peak areas was obtained and used to estimate the amount of ibuprofen released from the polymer composition. HPLC peaks of the ibuprofen released were the same than the non-extruded ibuprofen showing that ibuprofen is not degraded during the extrusion.

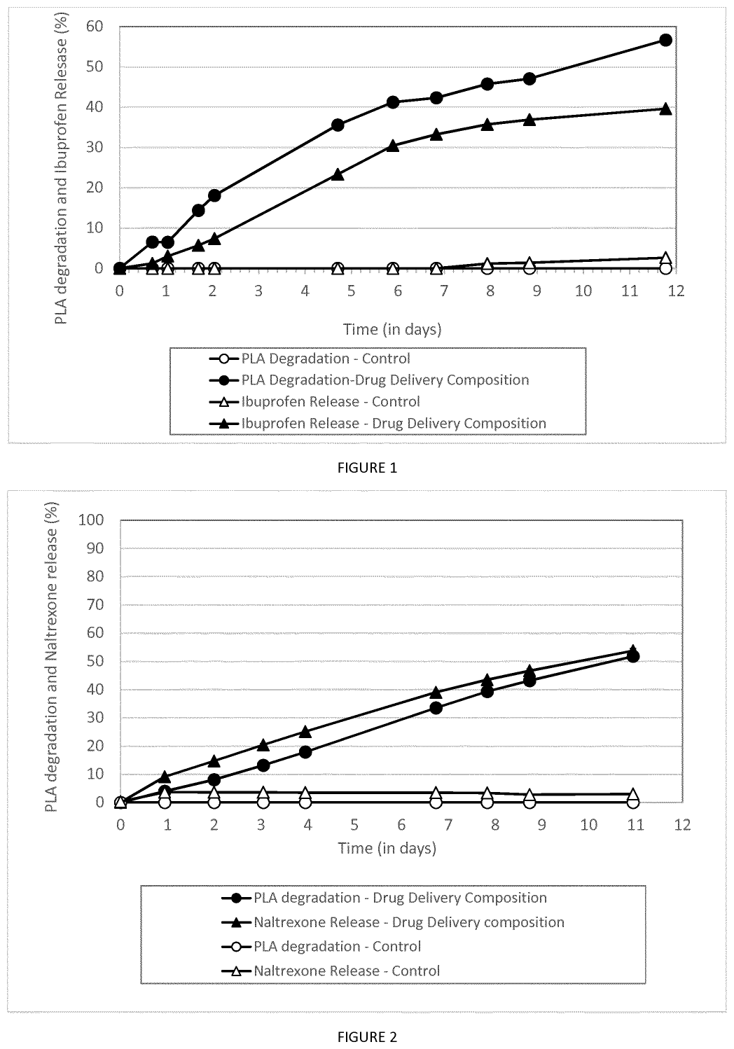

The results are shown in FIG. 1. PLA degradation is indicated in percentage (%) of the total lactic acid present in the PLA of the composition and the rate of ibuprofen release is indicated in % of the total ibuprofen embedded in the composition.

The results show that PLA is degraded only when the PLA-degrading enzyme is added in the composition, indicating that the PLA-degrading enzyme has maintained its PLA degradation activity in the drug delivery composition of the invention. The results also show that ibuprofen is not degraded through the extrusion processes. Thanks to the degradation of PLA by PLA-degrading enzyme, the ibuprofen is regularly released without any degradation by the enzyme. About 30% of ibuprofen (i.e. 0.15 grams) has been released in 6 days, corresponding to a daily dose of 25 mg. In the control composition without Savinase®, PLA was not degraded and ibuprofen was not significantly released.

The kinetics of PLA degradation can be adjusted thanks to the enzyme concentration and the kinetics of drug release could subsequently be controlled.

### Example 2—Drug Delivery Composition of the Invention Comprising Naltrexone, PLA and PLA-Degrading Enzyme

A drug delivery composition of the invention was prepared by mixing 87% by weight of micronized polymer of polylactic acid (Ingeo™ Biopolymer 4043D from NatureWorks, molecular weight (Mw) 180 000 g/mol), 8% by weight of naltrexone hydrochloride powder (from Sigma-Adrich) and 5% by weight of powder of Savinase® (prepared as Example 1), based on the total weight of the drug delivery composition. The mix was then extruded using a twin-screw extruder (Thermo Scientific HAAKE Minilab II) to incorporate simultaneously naltrexone and Savinase® into PLA. A control composition without Savinase® was also prepared. The twin screw extruder was used at 80 rpm and 168° C. with a manual loading of the composition.

Weight of each of the component (in grams) of the drug delivery composition and the control composition are summarized in Table 1.

TABLE 1
Composition in the drug delivery composition and the control.

| | Control | Drug delivery composition |
|---|---|---|
| PLA | 4.6 g | 4.35 g |
| Naltrexone | 0.4 g | 0.4 g |
| Hydrochloride | | |
| Savinase ® | 0 | 0.25 |

The degradation of the compositions was analyzed through the degradation of PLA and the release of naltrexone.

Compositions were cut in small fragments with a cutting pliers. About 100 mg of these compositions were introduced in a dialysis tubing cellulose membrane (cut off 14 000 Da—from Sigma-Aldrich) with 3 mL of Tris-HCl buffer 0.1 M pH 8. The dialysis tubings were then introduced in 50 mL of Tris-HCl buffer 0.1 M pH 8 and incubated at 37° C. during several days. Samples were taken off at different times during the degradation of the compositions.

The degradation of PLA and the release of naltrexone were analyzed by UHPLC by titration of lactic acid (as described in Example 1) and naltrexone (using method described below).

### UHPLC Method Used for Naltrexone Titration:

An Ultimate 3000 HPLC system (Thermofisher Scientific) equipped with Diode Array Detector (DAD-3000(RS)) and a Phenomenex Kinetex EVO C18, LC Column 100×2.1 mm, 2.6 μm with a pore size of 100 Å. The column was controlled to a temperature of 30° C. The mobile phase was a gradient of Ammonium Bicarbonate 20 mM pH9/Acetonitrile (95/5% to 35/65 in 5 min) with a flow rate of 0.75 mL/min. Naltrexone hydrochloride powder was accurately weighed and dissolved in water to give 450 μg/mL solution. Subsequent dilutions were made with water to get concentrations of 7-450 μg/mL. The standard solutions prepared as above were injected in the same conditions of samples. The peak areas of the naltrexone concentration were calculated. The regression of the naltrexone concentration over the peak areas was obtained and used to estimate the amount of naltrexone released from the polymer. HPLC peaks of the naltrexone released were the same than the non-extruded naltrexone showing that naltrexone is not degraded during the extrusion.

The results are shown in FIG. 2. PLA degradation is indicated in percentage (%) of the total lactic acid present in the PLA of the composition and naltrexone released is indicated in percentage (%) of the total naltrexone embedded in the composition.

The results show that PLA is degraded only when the PLA-degrading enzyme is added in the composition, indicating that the PLA-degrading enzyme has maintained its PLA degradation activity in the drug delivery composition of the invention. The results also show that naltrexone is not degraded through the extrusion processes. Thanks to the degradation of PLA by PLA-degrading enzyme, the naltrexone is regularly released without any degradation by the enzyme. About 54% of naltrexone (i.e. 0.22 grams) has been released in 11 days, corresponding to a daily dose of 20 mg. In the control composition without Savinase®, PLA was not degraded and naltrexone was not significantly released.

The kinetics of PLA degradation can be adjusted thanks to the enzyme concentration and the kinetics of drug release could subsequently be controlled.

### Example 3—Drug Delivery Composition of the Invention Comprising Estradiol, PLA and PLA-Degrading Enzyme

A drug delivery composition of the invention was prepared by mixing 90% by weight of micronized polymer of polylactic acid (Ingeo™ Biopolymer 4043D from NatureWorks, Mw 180 000 g/mol), 5% by weight of estradiol powder (from Sigma-Adrich) and 5% by weight of powder of Savinase® (prepared as Example 1), based on the total weight of the drug delivery composition. The mix was then extruded using a twin-screw extruder (Thermo Scientific HAAKE Minilab II) to incorporate estradiol and Savinase® into PLA. A control composition without Savinase® was also prepared. The twin screw extruder was used at 80 rpm and 165° C. with a manual loading of the composition.

Weight of each of the component (in grams) of the drug delivery composition and the control composition are summarized in Table 2.

TABLE 2
Composition of the drug delivery composition and the control.

| | Control | Drug delivery composition |
|---|---|---|
| PLA | 4.75 g | 4.5 g |
| Estradiol | 0.25 g | 0.25 g |
| Savinase ® | 0 | 0.25 g |

The degradation of the compositions obtained through the degradation of PLA and the release of estradiol were analyzed.

Compositions were cut in small fragments with a cutting pliers. About 50 mg of these compositions were introduced in a dialysis tubing cellulose membrane (cut off 14 000 Da-Sigma-Aldrich) with 3 mL of Tris-HCl buffer 0.1 M pH 8. The dialysis tubings were then introduced in 50 mL of Tris-HCl buffer 0.1 M pH 8 and incubated at 37° C. during several days. As many vials as sampling points was prepared because estradiol has a low solubility (around 3.6 mg/L). For each sampling point, a vial was used. 1 mL was taken off to titrate lactic acid and the rest of the sample was diluted in 52 mL of acetonitrile. If necessary additional dilutions were applicated.

The degradation of PLA and the release of estradiol were analyzed by UHPLC by titration of lactic acid (as described in Example 1) and estradiol (using method described below).

### UHPLC Method Used for Estradiol Titration:

An Ultimate 3000 HPLC system (Thermofisher Scientific) equipped with Diode Array Detector (DAD-3000(RS)) and a Phenomenex Kinetex EVO C18, LC Column 100×2.1 mm, 2.6 μm with a pore size of 100 Å. The column was controlled to a temperature of 30° C. The mobile phase was a gradient of Ammonium Bicarbonate 20 mM pH9/Acetonitrile (85/15% to 35/65 in 5 min) with a flow rate of 0.75 mL/min. Estradiol powder was accurately weighed and dissolved in 80% acetonitrile to give 110 μg/mL solution. Subsequent dilutions were made with water to get concentrations of 0.3-11 μg/mL. The standard solutions prepared as above were injected in the same conditions of samples. The peak areas of the estradiol concentration were calculated. The regression of the estradiol concentrations over the peak areas was obtained and used to estimate the amount of estradiol released from the polymer. HPLC peaks of the estradiol released were the same than the non-extruded estradiol showing that estradiol is not degraded during the extrusion.

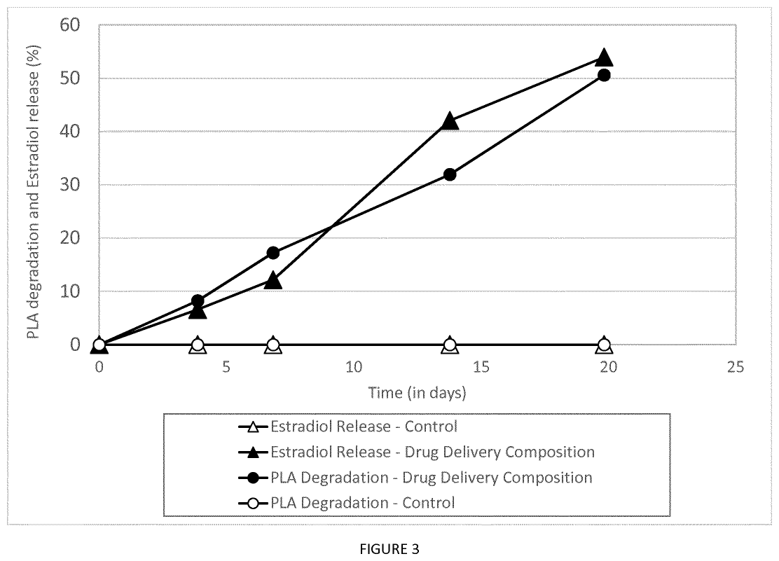

The results are shown in FIG. 3. PLA degradation is indicated in percentage (%) of the total lactic acid present in the PLA of the composition and estradiol release is indicated in percentage of the total estradiol embedded in the composition.

The results show that in the drug delivery composition of the invention, the release of Estradiol follows the degradation of PLA polymers by Savinase®. In the control composition without Savinase®, PLA was not degraded and estradiol was not released.

The results show that PLA is degraded only when the PLA-degrading enzyme is added in the composition, indicating that the PLA-degrading enzyme has maintained its PLA degradation activity in the drug delivery composition of the invention. The results also show that estradiol is not degraded through the extrusion processes. Thanks to the degradation of PLA by PLA-degrading enzyme, the estradiol is regularly released without any degradation by the enzyme. About 53% of estradiol has been released in 20 days, corresponding to a daily dose of 70 μg when considering a drug delivery composition of 50 mg. In the control composition without Savinase®, PLA was not degraded and estradiol was not significantly released.

### Example 4—Drug Delivery Composition of the Invention Comprising Lysozyme, PLA and PLA-Degrading Enzyme

A masterbatch comprising 50% by weight lysozyme and 50% by weight PolyCaprolactone (PCL, Capa™ 6500 from Perstorp, melting temperature between 58-60° C.) based on the total weight of the masterbatch was prepared by mixing 2.5 g of micronized PCL and 2.5 g of lysozyme powder (from Sigma-Aldrich, denaturation temperature of 76° C., 14.7 kDa). The mix was then extruded using a twin-screw extruder (Thermo Scientific HAAKE Minilab II) at 78° C., 80 Rpm with a manual loading.

A drug delivery composition of the invention was prepared by mixing 1 gram (20%) of said masterbatch cut in small fragments (around 2 mm×2 mm), 3.5 grams (70%) of micronized polymer of polylactic acid (Ingeo™ Biopolymer 4043D from NatureWorks, Mw 180000 g/mol), and 0.5 gram (10%) of Savinase® powder (see Example 1). The mix of the drug delivery composition was then extruded using the same extruder at 80 rpm and 165° C. with a manual loading of the composition.

The lysozyme was extracted from the drug delivery composition by liquid-liquid extraction. 50 mg of drug delivery composition were solubilized in 2.5 mL of Dichloromethane. Then 7.5 mL of cold 66 mM potassium phosphate buffer pH6.24 was added. The mix was vigorously vortex. Samples were maintained in ice between each step. After phase separation, aqueous phase was taken off and lysozyme activity was measured using Lysozyme activity Kit (from Sigma-Aldrich).

After two extrusions, at 78° C. and 165° C., the lysozyme embedded in the composition still exhibits activity (results not shown).

### Example 5—Incorporation of a High Quantity of Drug in a Polymer-Based Matrix by Extrusion

### Example 5.1—Incorporation of a High Quantity of Naltrexone in a Polymer-Based Matrix by Extrusion

A composition comprising 50% PLGA and 50% Naltrexone was prepared by mixing 2.5 grams of DL-Lactide/Glycolide copolymer (PLGA or PLA/PGA-PURASORB PDLG 5002A from Corbion Purac with a rubbery plateau) and 2.5 grams of Naltrexone hydrochloride powder (from Sigma-Adrich). The mix was then extruded using a twin-screw extruder (Thermo Scientific HAAKE Minilab II) to incorporate Naltrexone into PLGA. The twin screw extruder was used at 80 rpm and 100° C. with a manual loading of the composition.

The extruded composition is obtained in the form of solid pellets, suitable to be processed in a subsequent extrusion process and/or to be shaped to form a drug delivery device.

The composition has been cut in small fragments and 20% by weight of such composition has been mixed with 80% by weight with same copolymer of PLGA (PURASORB PDLG 5002A from Corbion Purac) to be submitted to extrusion at 100° C., 80 rpm, using the same extruder as described above. The resulting composition is also obtained in the form of solid pellets, suitable to be shaped to form a drug delivery device

The results show that it is possible to introduce about 50% of drug in a polymer composition by extrusion and to obtain a composition suitable to be subsequently processed or directly shaped to form a drug delivery device.

### Example 5.2—Incorporation of a High Quantity of Lysozyme in PCL by Extrusion

A composition comprising 50% PCL and 50% lysozyme was prepared by mixing 2.5 grams of PCL powder (Capa™ 6500 from Perstorp, melting temperature between 58-60° C.) and 2.5 grams of lysozyme powder (from Sigma-Aldrich, temperature of denaturation 74° C.). The mix was then extruded using a twin-screw extruder (Thermo Scientific HAAKE Minilab II) at 78° C., 80 Rpm with a manual loading of the composition.

The lysozyme was extracted from the drug delivery composition by liquid-liquid extraction as described in Example 4 and lysozyme activity was titrated with lysozyme activity kit (from Sigma-Aldrich). The results show that is possible to introduce about 50% of lysozyme in a polymer composition by extrusion, such drug retaining 95% activity after such extrusion.

### Example 5.3—Incorporation of a High Quantity of Lysozyme in PLA/PGA Copolymer by Extrusion

A composition comprising 50% of PLGA and 50% of lysozyme was prepared by mixing 2.5 grams of PLGA copolymer powder (PURASORB PDLG 5002A from Corbion Purac) and 2.5 grams of lysozyme powder (from Sigma-Aldrich). The mix was then extruded using a twin-screw extruder (Thermo Scientific HAAKE Minilab II) at 100° C., 80 Rpm with a manual loading of the composition.

The extruded composition is obtained in the form of solid pellets, suitable to be processed in a subsequent extrusion process and/or to be shaped to form a drug delivery device.

The composition has been cut in small fragments and 20% by weight of such composition has been mixed with 80% by weight with another copolymer of PLGA (PURASORB PDLG 5010 from Corbion purac) to be submitted to extrusion at 100° C., 80 rpm, using the same extruder as described above. The resulting composition is also obtained in the form of solid pellets, suitable to be shaped to form a drug delivery device

The results show that it is possible to introduce about 50% of a protein in a polymer composition by extrusion and to obtain a composition suitable to be subsequently processed or directly shaped to form a drug delivery device.

1-15. (canceled)

16. A drug delivery composition, wherein said composition comprises a drug and a polymer-degrading enzyme embedded into a polymer-based matrix, and wherein said composition is obtained by incorporation of said drug and said enzyme in said polymer-based matrix during heat treatment at a temperature T at which the polymer is in a partially or totally molten state.

17. The composition of claim 16, wherein said composition comprises:
from 50 to 99.98% by weight of polymer-based matrix,
from 0.01 to 49.99% by weight of the drug, and
from 0.01 to 30% by weight of the polymer-degrading enzyme.

18. The composition of claim 16, wherein the polymer-degrading enzyme is able to degrade at least one polymer of the polymer-based matrix.

19. The composition of claim 16, wherein the polymer-degrading enzyme is selected from proteases, esterases, cutinases, or lipases.

20. The composition of claim 16, wherein the polymer-based matrix contains at least one polymer selected from polylactic acid (PLA), polybutylene adipate terephthalate (PBAT), polyhydroxyalkanoate (PHA), polyglycolic acid (PGA), polybutylene succinate (PBS), polycaprolactone (PCL), poly(ethylene adipate) (PEA), dextrane, gelatin, poly butylene succinate adipate (PBSA), polydioxanone (PDS), polyethylene glycol (PEG), polyethylene oxide (PEO) or copolymers, and blends/mixtures thereof.

21. The composition of claim 16, wherein the polymer-based matrix contains at least PLA, PLLA and/or PDLA.

22. The composition of claim 16, wherein the polymer-based matrix comprises lactic acid copolymers, selected from PLA-based heteropolymers.

23. The composition of claim 22, wherein the PLA-based heteropolymers is selected from poly(lactic-co-glycolic acid) copolymers (PLA-co-PGA), poly(lactic-co-caprolactone) copolymers (PLA-co-PCL), poly(lactic-co-ethyleneglycol) copolymers (PLA-co-PEG), poly(lactic-co-ethylene oxide) copolymers (PLA-co-PEO) and grafted PLA (PLA-g-gelatine).

24. The composition of claim 16, wherein the polymer-based matrix contains PCL and/or wherein the polymer-based matrix contains PGA.

25. The composition of claim 16, wherein the drug is selected from chemicals, pharmaceutical compound, nutraceutical compound, amino acids, peptides, proteins, polysaccharides, lipid derivatives, antibiotics, analgesics, vaccines, vaccine adjuvants, anti-inflammatory agents, anti-tumor agents, hormones, cytokines, anti-fungal agents, anti-viral agents, anti-bacterial agents, anti-diabetics, steroids, vitamins, pro-vitamins, antioxidants, mineral salts, trace elements, specific enzyme inhibitor, growth stimulating agent, immunosuppressors, immuno-modulators, anti-hypertensive drugs, anti-arrhythmic drugs, inotropic drugs, addiction therapy drugs, anti-epileptic drugs, anti-aging drugs, drugs to treat neuropathies or pain, hypolipemic drugs, anti-coagulants, antibodies or antibody fragments, antigens, anti-depressant or psychotropic agents, neuro-modulators, drugs for treating a disease selected from brain disease, liver disease, pulmonary disease, cardiac disease, gastric disease, intestine disease, ovary disease, testis disease, urological disease, genital disease, bone disease, muscle disease, endometrial disease, pancreatic disease and/or renal disease, ophthalmic drugs, anti-allergic agents, contraceptive or luteinizing agents, enzymes, Traditional Chinese Medicines, nutrients, cosmetics and mixtures of at least two of these drugs.

26. The composition of claim 16, wherein the composition is a pharmaceutical composition.

27. The composition of claim 26, wherein the pharmaceutical composition is selected from a tablet, gel, coating, particles, and microbeads.

28. A drug delivery device obtained from the drug delivery composition of claim 16.

29. The drug delivery device according to claim 28, wherein the device is a medical device.

30. The drug delivery device according to claim 29, wherein the medical device is selected from an implant, film, stent, leaflet, valve, coil, scaffold, dressing, rod, patch, fibers, suture fibers, screw, bone plate or implant, bone cement and prostheses.

31. A process for preparing a drug delivery composition, wherein said composition comprises a polymer-based matrix, a drug, and a polymer-degrading enzyme, and wherein said process comprises incorporating said drug and said enzyme into said polymer-based matrix during heat treatment of the polymer at a temperature T at which the polymer is in a partially or totally molten state.

32. The process of claim 31, wherein the drug and enzyme are incorporated at a temperature T between 50° C. and 200° C.

33. The process of claim 31, wherein the heat treatment is selected from extrusion, internal mixing, co-kneading, injection-molding, thermoforming, rotary molding, compression, calendering, ironing, coating, stratification, expansion, pultrusion, extrusion blow-molding, extrusion-swelling, compression-granulation and 3D printing.

34. A medical drug delivery device obtainable by the process of claim 31.
